(19) United States
(12) Patent Application Publication
(10) Pub. No.: US 2013/0171618 A1
(43) Pub. Date: Jul. 4, 2013

(54) POROUS MEMBRANES HAVING A POLYMERIC COATING AND METHODS FOR THEIR PREPARATION AND USE

(71) Applicants: Bing Li, Troy, NY (US); Cathryn Ellen Olsen, Wilton, NY (US); David Roger Moore, Rexford, NY (US)

(72) Inventors: Bing Li, Troy, NY (US); Cathryn Ellen Olsen, Wilton, NY (US); David Roger Moore, Rexford, NY (US)

(73) Assignee: GENERAL ELECTRIC COMPANY, SCHENECTADY, NY (US)

(21) Appl. No.: 13/340,052

(22) Filed: Dec. 29, 2011

(51) Int. Cl. G01N 33/53

(52) U.S. Cl. CPC G01N 33/5306
USPC 435/5; 436/501; 435/7.9; 435/7.92; 435/7.1

(57) ABSTRACT

A modified porous membrane comprising a polymer coating grafted to a porous membrane is described. The polymer coatings grafted to the porous membranes generally comprise a polymer of variable length of an electron beam (e-beam) reactive moiety, designated “poly-(A)ₓ,” a linkage group that forms a bond between the between the poly-(A)ₓ, and a functional B group available to react with a chemical group on a biomolecule, wherein the polymer coating on the porous membrane facilitates the immobilization of a biomolecule, such as DNA, RNA, a protein, and an antibody, on the porous membrane. The compositions find use in immunoassays, in vitro diagnostic tests, point of care tests, techniques for the isolation of a biomolecule from a biological sample, and other methods that require the immobilization of a biomolecule on a porous membrane. Methods of making these modified porous membranes are also disclosed.

### FIELD OF THE INVENTION

The present disclosure generally relates to porous membranes permanently grafted with a polymeric coating to facilitate the immobilization of a biomolecule on the porous membrane. Methods of preparing and using the modified porous membranes with these polymeric coatings are also described.

### BACKGROUND

Porous membranes, such as nitrocellulose membranes, are routinely used in a variety of processes, including biological applications that require the immobilization of one or more biomolecules. These biomolecules include but are not limited to proteins (e.g., antibodies) and nucleic acids (e.g., deoxyribonucleic acid (DNA) and ribonucleic acid (RNA)). Membranes are needed for the immobilization of biomolecules for use in, for example, immunoassays, in vitro diagnostic tests, particularly point-of-care diagnostic methods, and separation of analytes or biomolecules in biological samples (e.g., blood, urine, saliva, sputum, other bodily secretions, cells, and tissue samples) for a variety of biological processes and medical techniques.

Nitrocellulose membranes exhibit an essentially non-specific interaction between the nitrocellulose membrane and biomolecule(s), and researchers have traditionally relied upon this passive association as the basis for the use of nitrocellulose membranes in a variety of “entrapment” type immobilization methods. Reliance on this passive interaction between the nitrocellulose membrane and a biomolecule of interest, however, may lead to complications for successfully using nitrocellulose membranes in many biological applications because it necessarily limits the amount of the biomolecule that can be immobilized on the nitrocellulose membrane. Dependence on this passive binding process, while perhaps sufficient for certain applications in which an analyte or biomolecule is present in a high enough concentration in a biological sample to be analyzed, this reliance on these passive, physical absorption properties limits traditional nitrocellulose membrane-based techniques for example, in disease states in which the analyte or biomolecule quantity is low and possibly “undetectable” by known compositions and standard methodologies.

Previous research has utilized various techniques to modify porous membranes, for example, nitrocellulose membranes, to improve binding or immobilization of biomolecules on porous membrane substrates. Methods to promote binding of biomolecules to porous membranes, include but are not limited to, ammonia plasma treatment, oxygen plasma treatment, covalent bonding of “bridging” molecules, and hydroxylamine treatment of nitrocellulose membranes. These techniques and membrane modifications have not achieved the desired goals of those of skill in the art.

New methods of modifying (e.g., chemically modifying) porous membranes to improve immobilization and binding of biomolecules (e.g., proteins and nucleic acids) of interest to porous membrane substrates are needed in the art. Such modified porous membranes, including modified nitrocellulose membranes, would find use in, for example, immunoassays, in vitro diagnostic tests (e.g., point-of-care diagnostic applications), and techniques for the separation of biomolecules of interest in biological samples. Moreover, modified porous membranes would allow for increased biomolecule (e.g., DNA, RNA, and protein, particularly an antibody) binding to the porous membrane, thereby leading to improved specificity and sensitivity of immunoassays and diagnostic tests, a reduced number of false positive and false negative test results, a reduction in the concentration of an analyte or a biomolecule in a biological sample needed for minimum, accurate biomolecule detection in, for example, immunoassays and point-of-care diagnostics, particularly those that detect analytes and biomolecules that are present even in biological samples in small quantities. Modified porous membranes of the application would further shorten the time needed to accurately detect the presence of a biomolecule, thereby also providing faster positive or negative test results. Accordingly, it would be advantageous to provide porous membranes having polymer coatings that improve the immobilization and binding of biomolecules to these porous membranes. Modified porous membranes may be further generated by novel methods of production.

### BRIEF DESCRIPTION

Modified porous membranes are described herein. In a particular embodiment, the porous membrane comprises a coating of at least one polymer grafted to the porous membrane. The polymer coating is generally permanently (e.g., covalently) bound to the porous membrane. In certain aspects of the invention, the porous membrane is a nitrocellulose membrane. The polymer may be grafted to the porous membrane by any method, including the generation of free radicals such as by derivatizing an electron beam (e-beam) reactive moiety, wherein the e-beam reactive moiety permanently bonds the polymer coating to the porous membrane upon exposure to e-beam irradiation. Free radicals may alternatively be generated for the disclosed compositions and methods by including, without limitation, ultraviolet irradiation, gamma irradiation, corona discharge, and the use of a chemical initiator.

In a further embodiment, the modified porous membrane disclosed herein comprises a polymer coating of at least one epoxy group-containing compound. The epoxy group-containing compound is grafted to the porous membrane by generating free radicals on the porous membrane using e-beam irradiation and a polymer of variable length that contains an e-beam reactive moiety, designated “poly-(A)ₓ polymer,” a “linkage” that forms a bond between the poly-(A)ₓ polymer, and a functional “B” group that is able to react with a chemical group present on a biomolecule, thereby resulting in the formation of a polymer coating of, for example, an epoxy group-containing compound, such as GMA, permanently bound to the porous membrane.

The compositions herein include modified porous membranes that comprise a polymer coating of, for example, a polymer of an epoxy group-containing compound permanently grafted to a porous membrane. In certain embodiments, the modified porous membranes comprise polymers of the epoxy group-containing molecule glycidal methylacrylate (GMA). The compositions find use in methods for improving the binding of biomolecules, such as proteins (e.g., antibodies) and nucleic acids (e.g., DNA or RNA), to porous membranes, including but not limited to, nitrocellulose membranes. In particular aspects, the compositions are utilized in immunoassays, in vitro diagnostic tests, techniques for the identification or isolation of biomolecules of interest from biological samples (e.g., blood, urine, saliva, sputum, and samplings of cells or tissues), and various other biological methods that require the immobilization of a biomolecule on a porous membrane substrate.

### DRAWINGS

These and other features, aspects, and advantages of the chemically modified porous membranes will become better understood when the following detailed description is read with reference to the accompanying drawings in which like characters represent like parts throughout the drawings, wherein:

FIG. 1 provides the general formula (Formula (I)) of the modified porous membranes described herein. The details of the specific components of the general formula are provided throughout the specification.

FIG. 2 is a schematic representation of the mechanism of grafting glycidal methylacrylate (GMA) on a nitrocellulose membrane by e-beam irradiation.

FIG. 3 provides the ATR FTIR spectra obtained for the various reactants used to graft GMA onto nitrocellulose membranes in accordance with exemplary methods of the methods of the invention. Specifically, the spectra for nitrocellulose, nitrocellulose+water, nitrocellulose+water+Tween-20™ (polyoxyethylene (20) sorbitan monolaurate), nitrocellulose+water+GMA+Tween-20™ (polyoxyethylene (20) sorbitan monolaurate), GMA, and Tween-20™ (polyoxyethylene (20) sorbitan monolaurate) are presented in FIG. 2. See Example 1 below for details of the method used.

FIG. 4 provides the ATR FTIR spectra for replicate samples of nitrocellulose grafted with GMA using either 10 kGy or 50 kGy of e-beam radiation. Additional details are described in Example 1.

FIG. 5 provides the results from ¹H DOSY NMR analysis of acetone dissolved unmodified nitrocellulose membranes (A) and modified nitrocellulose membranes grafted with GMA using either 10 kGy (B) or 50 kGy (C) of e-beam radiation. See Example 1 for further details.

FIG. 6 sets forth the results of fluorescence scanning and colorimetric analysis of unmodified and modified nitrocellulose membranes utilized in assays to assess BSA protein binding to these membranes. The assays were performed under different reaction conditions as described below in Example 2.

FIG. 7 provides the photographic results of a model lateral flow assay (e.g., a pregnancy test) performed using either unmodified or modified nitrocellulose membranes grafted with GMA. This example is described in Example 3.

FIG. 8 demonstrates that a significantly lower detection limit for the antigen human chorionic gonadotropin (hCG) is obtained in lateral flow assays utilizing modified nitrocellulose membrane strips grafted with GMA as compared to results obtained in corresponding examples with unmodified nitrocellulose membrane strips. See Example 5 below for additional details.

FIG. 9 provides a graph demonstrating a significant decrease in the time to detect equivalent amounts of hCG in lateral flow assays utilizing modified nitrocellulose membrane strips grafted with GMA as compared to results obtained in corresponding examples with unmodified nitrocellulose membrane strips. A visual positive result for the presence of hCG in a lateral flow assay with GMA-grafted nitrocellulose was observed at approximately 2 minutes versus the roughly 20 minutes necessary to achieve similar results in a lateral flow assay with using an unmodified nitrocellulose membrane in an identical lateral flow assay. See Example 6 additional details.

### DETAILED DESCRIPTION

Modified porous membranes are provided herein that comprise at least one polymer coating grafted to the porous membrane to facilitate immobilization of a biomolecule on the porous membrane. The term “modified” as used herein, particularly in reference to the disclosed porous membranes is intended to include any alteration to the porous membrane, for example, a chemical alteration, of the original, unmodified porous membrane. The “modified” porous membranes of the invention may be a chemically modified porous membranes comprising a polymeric coating comprising, for example, an epoxy group-containing compound (e.g., GMA), grafted to the porous membrane. In one aspect of the disclosure, the porous membrane is a nitrocellulose membrane comprising polymers of the epoxy group-containing compound is GMA grafted on the porous membrane.

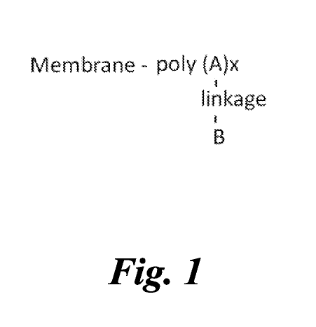

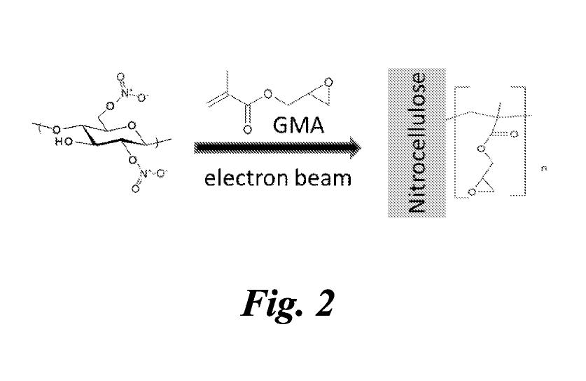

A schematic of exemplary modified porous membranes is provided below and set forth in FIG. 1 and FIG. 2. As shown in the diagrams, in certain aspects, the porous membrane of this disclosure has the structure of Formula (I) that includes a polymer coating bound to a porous membrane, wherein the polymer coating comprises: 1) a polymer of a variable length of a chain monomers of an electron (e-beam) reactive moiety (designated as poly(A)ₓ; 2) a linkage that forms a bond between (poly(A)ₓ); and 3) a functional group labeled B group which facilitates reaction with chemical groups, for example, an amine group present on a biomolecule of interest, thereby facilitating immobilization of a biomolecule on the porous membrane. The polymer coating (e.g., labeled “poly(A)ₓ-linkage-B” in the schematic below), comprises several components (e.g., poly(A)ₓ polymer, a linkage, and a functional moiety B) is grafted (e.g., covalently bond) the polymeric coating to a porous membrane. See below and FIG. 1 and FIG. 2 for a more detailed description of the components and functions of the polymer coating.

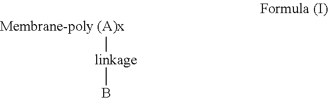

To more clearly and concisely describe and point out the subject matter of the claimed invention, the following definitions are provided for specific terms, which are used in the following description and in the appended claims. Throughout the specification, exemplification of specific terms should be considered as non-limiting examples.

The term “modified” as used herein, particularly in reference to disclosed porous membranes and solid phase materials, is intended to include any alteration to a porous membrane or a solid phase material, for example, a chemical alteration, of the original, unmodified porous membrane or solid phase membrane substrate. The “modified” porous membranes of the invention may be modified (e.g., chemically modified) porous membranes comprising polymers, such as an epoxy group-containing compound, grafted to the porous membrane. In one aspect of the disclosure, the porous membrane is a nitrocellulose membrane and the epoxy group-containing compound is GMA.

Methods for preparing the porous membranes having a polymer coating permanently grafted on the porous membranes are further provided. In some embodiments, a modified porous membrane is grafted with a polymer coating by first immersing the porous membrane in a solution of a polymer of variable length comprising an e-beam reactive moiety (e.g., “poly-(A)x polymer”), a “linkage” that forms a bond between the poly-(A)x polymer, and a functional B group available to react with a functional moiety present on a biomolecule (see below and FIG. 1) and then subjected to e-beam. For example, a porous membrane is immersed in an epoxy group-containing compound (e.g., GMA) and then subjected to e-beam irradiation. Alternatively, in other aspects of the invention, the modified porous membranes are prepared by first subjecting a porous membrane to e-beam irradiation followed by immersing the membrane in a solution of, for example, an epoxy group-containing compound such as GMA, as described above. The methods of production of the modified porous membrane substrates described herein that vary, for example, the ordering of the method steps of immersing and the e-beam irradiation step are encompassed by the instant disclosure.

When used in the context of a method for preparing a modified porous membrane as described in greater detail below, the term “immersing” the porous membrane in a solution of, for example, an epoxy group-containing compound such as GMA, as recited in the claims, is generally accomplished by dipping the entire porous membrane in the polymeric coating (poly(A)ₓ-linkage-B) solution and then removing any excess solution.

Various porous membranes are encompassed by this disclosure. For exemplary purposes only and without any limitation intended, the unmodified porous membrane may include a nitrocellulose membrane, a cellulose membrane, a cellulose acetate membrane, a regenerated cellulose membrane, a nitrocellulose mixed ester membrane, a polyethersulfone membrane, a nylon membrane, a polyolefin membrane, a polyester membrane, a polycarbonate membrane, a polypropylene membrane, a polyvinylidene difluoride membrane, a polyethylene membrane, a polystyrene membrane, a polyurethane membrane, a polyphenylene oxide membrane, a poly(tetrafluoroethylene-co-hexafluoropropylene membrane, or any combination of two or more of the above porous membranes.

Nitrocellulose membranes are currently widely used in a variety of biological applications that require the immobilization of a particular biomolecule (e.g., DNA, RNA, or a protein such as an antibody) on a solid phase material. “Porous membrane” is intended to refer to, without limitation, to any porous membrane, including any commercially available porous membrane, particularly a commercially available nitrocellulose membrane. In certain aspects of this disclosure, a nitrocellulose membrane is chemically modified to comprise, as set forth in FIG. 1, a polymeric coating that facilitates biomolecule immobilization on a porous modified membrane. For example, one such modified porous membrane comprises an epoxy group-containing compound (e.g., GMA) grafted to a nitrocellulose membrane. Nitrocellulose membranes, which are made of a nitrocellulose polymer, have a strong affinity for DNA, RNA, and protein and prevent the denaturation of such biomolecules.

“Nitrocellulose membranes” as used in this application include all those porous membrane products containing any nitrogen concentration, a diversity of pore sizes, and variable membrane thicknesses. In particular embodiments, the pore size of the porous membrane may be in the range of 0.01 to 50 microns. Moreover, pore diameter may be uniform throughout the porous membrane or, alternatively, pore diameter may be irregular. It is well within the skill and the knowledge of one in the art to select a porous membrane, such as a nitrocellulose membrane, with the appropriate nitrogen content, pore size, and membrane thickness to achieve a specific, desired result. Moreover, the skilled artisan would immediately understand and appreciate the meaning of the phrase a “nitrocellulose membrane” and that such nitrocellulose membranes, for example, or commercially available nitrocellulose membranes, may be “unbacked” membranes or alternatively contain a “backing material” or “backing support” such as a polyester (PE). The choice as to whether to use an “unbacked” or “backed” porous membrane (e.g., nitrocellulose membrane) is dependent upon the particular application to be performed and is well within the purview of one of ordinary skill in the art to make such a selection.

Nitrocellulose membranes, having any nitrogen concentration, pore size, or the presence or absence of a backing support are all encompassed in the term “nitrocellulose membrane” as used herein. Nitrocellulose membranes have a variety of chemical and physical properties and are routinely used in biological techniques that require, for example, the immobilization of a biomolecule of interest (e.g., an antibody) to a porous membrane or for the collection of biomolecules on these porous membranes in order to separate them from other proteins, nucleic acids, and biomolecules or the like in a biological sample to be analyzed. Any nitrocellulose membrane may be utilized in the present disclosure.

Although porous membranes are referred to throughout the instant application, the compositions, methods of preparation, and methods of use are equally applicable to other solid phase materials useful in the immobilization of a biomolecule, as recited in the claims herein. Such solid phase materials include but are not limited to glass beads, glass fibres, latex beads, nodes, cakes, nanoparticles, hollow membrane tubes, and any combination of two or more of the above solid phase materials.

Without intending to be limited to a particular mechanism of action, as used in this disclosure, the term “e-beam reactive moiety,” designated as “A” in FIG. 1 refers to any chemical functional group that is believed to self-polymerize when subjected to e-beam irradiation (e.g., poly(A)x in FIG. 2). Exemplary e-beam reactive moieties include but are not limited to those compounds that comprise a methacrylate, an acrylate, an acrylamide, a vinyl ketone, a styrenic, a vinyl ether, a vinyl-containing moiety, an allyl-containing moiety, a benzyl-based compound, and a tertiary-carbon (CHR3)-based compound, or two or more of the e-beam reactive moieties set forth above. Moreover, one of skill in the art could envision other appropriate e-beam reactive moieties for use in the invention based on this representative list.

The “linkage” shown in FIG. 1 that forms a bond between the poly(A)ₓ polymer and the functional B group, described below, includes but is not limited to an ester, an aliphatic, an aromatic, a hydrophilic compound, a hetero-aromatic compound, or any combination of two or more of these exemplary linkages.

The B functional group as labeled in the schematic presented in FIG. 1 includes, without intending to be limited in any way, an epoxy group-containing compound, a polyethylene glycol (PEG), an alkyne group, a hydroxyl group, an amine group, a halogen group, a tosyl group, a mesyl group, an azido group, an isocyanate group, an silane group, disilazanes, sulthydryls, carboxylates, isonitriles, phosphoramidites, nitrenes, hydrosilyl, nitrile, alkylphosphonates, and any combination of two or more of these functional moieties. While not meant to be limited to a particular mechanism of action, the B functional group may be introduced on the porous membrane through e-beam irradiation leading to the self-polymerization of the e-beam reactive moiety, which in turn makes the B functional group (e.g., an epoxy group) available to react with functional moieties, for example, an amine group present on a biomolecule, such as a protein, particularly an antibody, thereby facilitating immobilization of the biomolecule on the modified porous membrane. This modification is beneficial as many porous membranes, such as nitrocellulose membranes, lack the organic functional groups necessary to effectively bond to a porous membrane a biomolecule of interest that possesses, for example, an amino group(s) (e.g., proteins, more particularly antibodies).

An “epoxy group-containing compound” refers to any chemical compound that comprises at least one epoxy group. Any epoxy group-containing compound, such as GMA, may be used in the compositions and methods of this disclosure. In one embodiment, the modified porous membrane is a nitrocellulose membrane grafted with polymers of GMA.

In particular embodiments, the B functional group is an epoxy group-containing compound, for example an epoxy group. In these aspects of the disclosure the porous membrane (e.g., a nitrocellulose membrane) may comprise polymers of a compound such as GMA.

The term “biological sample” includes but is not limited to blood, serum, lymph, saliva, mucus, urine, other bodily secretions, cells, and tissue sections obtained from a human or non-human organism. Biological samples may be obtained by an individual undergoing the diagnostic test herself (e.g., blood glucose monitoring) or by a trained medical professional through a variety of techniques including, for example, aspirating blood using a needle or scraping or swabbing a particular area, such as a lesion on a patient's skin. Methods for collecting various biological samples are well known in the art.

“Immunoassay” is used herein in its broadest sense to include any technique based on the interaction between an antibody and its corresponding antigen. Such assays are based on the unique ability of an antibody to bind with high specificity to one or a very limited group of similar molecules (e.g., antigens). A molecule that binds to an antibody is called an antigen. Immunoassays can be carried out using either the antigen or antibody as the “capture” molecule to “entrap” the other member of the antibody-antigen pairing.

An exemplary, albeit not exhaustive list of immunoassays includes a lateral flow assay (e.g., a home pregnancy test), a radioimmunoassay (RIA), an enzyme immunoassay (EIA), an enzyme-linked immunosorbent assay (ELISA), a fluorescent immunoassay, and a chemiluminescent immunoassay. The skilled artisan in the field possesses the skills needed to select and implement the appropriate method(s) for a particular situation, as well as the techniques for performing these immunoassays, as well as the skills to interpret the results. Immunoassays may produce qualitative or quantitative results depending on the particular method of detection selected.

The lateral flow assay is a common immunoassay, largely due to its ease of use, and includes such products as commercially available home-pregnancy tests and routine drug tests. Lateral flow assays are particularly advantageous because the devices and methods are generally simple to use and to interpret the test results, even by an individual with no formal medical training. Lateral flow devices and methods are intended to detect the presence or absence of a target analyte or biomolecule (e.g., human chorionic gonadotropin (hCG) in a lateral flow home pregnancy test) in a biological sample (e.g., urine). Although there is variation among lateral flow devices and assays, these tests are commonly used for home testing, point of care testing, and laboratory use. Lateral flow assays are often presented in a convenient “dipstick” format, as described in the examples below, in which the biological sample to be tested flows along a solid substrate (e.g., a porous membrane, often a nitrocellulose membrane) via capillary action. In certain formats of lateral flow assays, the dipstick is immersed in the biological sample, it encounters one or more reagents previously imprinted on the dipstick as the biological sample flows up the test strip, thereby encountering lines or zones on the test strip that have been previously imprinted with, for example, an antibody or antigen (e.g., hCG). When the biological sample encounters this reagent(s), a signal is generated to indicate whether the test is positive or negative for the presence of the analyte or biomolecule of interest (e.g., frequently a line visible to the naked eye as in the detection of hCG in a home pregnancy test indicative of the presence of hCG in the patient's urine).

Lateral flow devices and methods are well known in the art. See, for example, U.S. Pat. Nos. 4,094,647; 4,313,734; 4,857,453; 5,073,484; 5,559,041; 5,571,726; 5,578,577; 5,591,645; 6,187,598; 6,352,862; and 6,485,982; all of which are herein incorporated by reference in their entirety. Inclusion of a modified porous membrane of this disclosure, such as a modified nitrocellulose membrane comprising a polymeric coating of GMA polymers, in known, for example, lateral flow devices and assays would significantly improve the performance, sensitivity, and specificity of such lateral flows devices and immunoassays, decrease the concentration of the analyte or biomolecule needed to obtain an accurate test results, and reduce the time to detect the presence or absence of the analyte or biomolecule, thereby minimizing the time required to acquire the test result.

All antibodies are proteins, more specifically glycoproteins, and exhibit binding specificity to an antigen (e.g., a portion of a polypeptide) of interest. The term “antibody” is used in the broadest sense and covers fully assembled antibodies, antibody fragments that can bind antigen (e.g., Fab′, F′(ab)₂, Fv, single chain antibodies, diabodies), and recombinant peptides comprising the foregoing. “Antibody fragments” comprise a portion of an intact antibody, preferably the antigen-binding or variable region of the intact antibody. Examples of antibody fragments include Fab, Fab′, F(ab′)₂, and Fv fragments, diabodies, and linear antibodies (Zapata et al. (1995) Protein Eng. 8(10):1057 1062), single-chain antibody molecules, and multi-specific antibodies formed from antibody fragments. Any antibody or antibody fragment may be used in the practice of the invention.

In certain aspects of this invention, detection of antibody binding or immobilization on a solid phase material, including but not limited to a nitrocellulose membrane, is needed. Any method known in the art for detecting antibody binding to a nitrocellulose membrane is encompassed by the disclosed invention. The determination and optimization of appropriate antibody binding detection techniques is standard and well within the routine capabilities of one of skill in the art. In some embodiments, detection of antibody binding can be facilitated by coupling the antibody to a detectable substance. Examples of detectable substances include various enzymes, prosthetic groups, fluorescent materials, luminescent materials, bioluminescent materials, and radioactive materials. Exemplary suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate (FITC), rhodamine, dichlorotriazinylamine fluorescein, dansyl chloride or phycoerythrin; a detectable luminescent material that may be couple to an antibody includes but is not limited to luminol; examples of bioluminescent materials include luciferase, luciferin, and aequorin; and examples of suitable radioactive material for detection of antibody binding include ¹²⁵I, ¹³¹I, ³⁵S, or ³H.

The modified porous membranes comprising a polymer coating of, for example, GMA may be further modified to comprise a hydrophilic compound immobilized on the porous membrane. The introduction of a hydrophilic compound onto the modified porous membrane comprising a polymeric coating may act as a blocking agent to decrease non-specific, background binding to the porous membrane (e.g., nitrocellulose membrane). Minimizing non-specific, background binding to a porous membrane improves the signal to noise ratio in, for example, immunoassays based on the specific interaction of an antibody immobilized on the porous membrane and a specific biomolecule of interest (e.g., a protein) in a sample being analyzed for the presence or quantity of this biomolecule.

In certain aspects of the invention, the claimed modified porous membranes, particularly nitrocellulose membranes, are prepared as described above using an aqueous solution of a polymeric coating described herein (poly(A)ₓ-linkage-B) (e.g., GMA). The solution of GMA may further comprise a co-solvent to improve the solubility of the GMA in water. For example, a surfactant, more particularly a non-ionic surfactant (e.g., polyoxyethylene (20) sorbitan monolaurate (Tween-20™)), may be used as a co-solvent to increase solubility of, for example, GMA, in water. One of skill in the art will appreciate that the appropriate amount of a particular co-solvent (e.g., a nonionic surfactant such as Tween-20™) needed to increase solubility of, for example, an epoxy group-containing compound must be determined and optimized experimentally.

Without intending to be limited to a particular method of making the modified porous membranes grafted with a polymeric coating, for example, GMA, exemplary methods of making the modified porous membranes are provided herein. Other methods may also be used to produce the modified porous membranes. In one embodiment, the modified porous membranes are prepared by providing a porous membrane; immersing the porous membrane in a solution of poly(A)ₓ-linkage-B (e.g., a compound such as GMA); subjecting the resultant porous membrane to e-beam irradiation; drying the porous membrane, and thereby preparing a modified porous membrane. Alternatively, the modified porous membranes may be prepared by first subjecting the porous membrane to e-beam irradiation and then immersing the porous membrane in a solution of a poly(A)ₓ-linkage-B, such as GMA. That is, the modified porous membranes of the invention may be first prepared by providing a porous membrane; subjecting the porous membrane to e-beam radiation; immersing the nitrocellulose membrane in a solution of a poly(A)ₓ-linkage-B (e.g., GMA); drying the porous membrane and thereby preparing a modified porous membrane.

Without intending to be limited to a particular mechanism, in the methods described above for producing a modified porous membrane, particularly a nitrocellulose membrane, e-beam radiation is believed to generate free radicals on the porous membrane which are then available to attack a double bond on, for example, the epoxy group-containing compound (e.g., GMA), thereby initiating self-polymerization of the epoxy group-containing compound, and resulting in grafting of a polymer coating on the porous membrane, particularly a nitrocellulose membrane. See FIG. 1 and FIG. 2. The functional group B grafted to the porous membrane (e.g., an epoxy group) are then available to react with amine and other chemical groups present on a biomolecule of interest, leading to increased binding of the biomolecule to the modified porous membrane. Increased specific binding of a biomolecule, such as an antibody, can improve the sensitivity and specificity of, for example, immunoassays.

The dosage of e-beam radiation used in the methods of grafting a polymer coating onto a porous membrane is selected to maximize the amount of the polymer coating that is grafted to the porous membrane while also limiting degradation of the porous membrane (e.g., nitrocellulose membrane) known to result from e-beam irradiation. One of skill in the art will recognize that the appropriate dose of e-beam radiation used in the preparation of the modified porous membranes of the invention will need to be optimized experimentally. In particular embodiments, the dose of e-beam radiation used in the methods to prepare a modified porous membrane may be in the range of less than 1 kGy to approximately 50 kGy. The design of assays to optimize parameters such as the amount of the polymer coating, co-solvent, and the dose of e-beam radiation appropriate for use in the methods of the invention is standard and well within the routine capabilities of those of skill in the art.

The modified porous membranes of the invention find use in various biological applications that are dependent upon the immobilization of a biomolecule on a porous membrane (e.g., a nitrocellulose membrane), including but not limited to immunoassays, in vitro diagnostic tests, and techniques for the isolation of a biomolecule of interest. Nitrocellulose membranes are of particular use in biological techniques because of their unique ability to immobilize nucleic acids (e.g., DNA and RNA) for use in Southern and Northern blots and for their binding affinity for amino acids (e.g., protein). As a result of these properties, nitrocellulose membranes are widely used as the substrate in diagnostic tests wherein antigen-antibody binding provides the test result (e.g., home pregnancy tests).

Although the ability of unmodified nitrocellulose membranes to bind biomolecules such as nucleic acids and proteins is beneficial, the modification of porous membranes, particularly nitrocellulose membranes, to facilitate the immobilization of biomolecules (e.g., DNA, RNA, and protein, more particularly an antibody), provides significant advantages over the binding of these biomolecules to unmodified porous membranes (e.g., nitrocellulose membranes).

Accordingly, in certain aspects of the invention, methods for improving the immobilization of a biomolecule on a porous membrane are described herein. Specifically, in certain embodiments, a method for improving immobilization of a biomolecule on a porous membrane comprises providing a modified porous membrane comprising a polymer coating as disclosed herein, incubating the modified porous membrane in a solution of a biomolecule, washing the porous membrane to remove unbound material, and thereby improving immobilization of the biomolecule to the porous membrane. The porous membrane may be washed in an aqueous solution comprising a surfactant, particularly a non-ionic surfactant, more particularly Tween-20™ (polyoxyethylene (20) sorbitan monolaurate), to further minimize non-specific binding to the modified porous membrane. In some embodiments, the biomolecule immobilized on the modified porous membrane, particularly a nitrocellulose membrane, is DNA, RNA, or a protein, such as an antibody.

Methods for improving the sensitivity of an immunoassay are also described herein comprising providing a modified porous membrane comprising the polymer coating described in detail herein (e.g., a polymer coating of GMA), incubating the modified porous membrane in a solution of a first antibody that specifically binds to an antigen, thereby resulting in immobilization of the antibody on the modified porous membrane, washing the modified porous membrane to remove excess, non-immobilized antibody, incubating the modified porous membrane comprising the immobilized antibody in a biological sample that may contain the analyte (e.g., antigen) that specifically binds to the immobilized antibody on the modified nitrocellulose membrane, and detecting binding of the antigen in the biological sample to the antibody immobilized on the modified porous membrane.

Alternatively, the biological sample may be first incubated with a second antibody that also specifically binds to the antigen of interest, wherein the second antibody is conjugated to a detectable substance. Such detectable substances include but are not limited to an enzyme, a prosthetic group, a fluorescent dye, a luminescent material, a bioluminescent material, a radioactive material, or gold particles. The biological sample pre-incubated with an antibody conjugated to a detectable substance is then incubated with a modified porous membrane comprising a polymer coating of, for example, GMA. The presence of the detectable substance on the antibody pre-incubated with the biological sample permits detection of the antigen in the biological sample being analyzed.

The methods described above wherein a chemically modified porous membrane (e.g., a nitrocellulose membrane) that possesses improved ability to bind a biomolecule such as DNA, RNA, or a protein imparts a number of advantages on immunoassays that utilize these modified porous membranes. For example, increased antibody immobilization on the modified porous membrane, reduces the amount of antibody needed to detect the presence of an antigen of interest, improved “capture” of the antigen from the biological sample because of the increased amount of antibody immobilized on the modified porous (e.g., a nitrocellulose membrane), leading to an increase in the antigen bound to the immobilized antibody, and a reduced amount of antibody in the biological sample to detect the presence of the biomolecule in the biological sample.

A variety of immunoassays exist in the art, including those for drug testing, hormones, numerous disease-related proteins, tumor protein markers, and protein markers for cardiac injury. Immunoassays are also used to detect antigens on infectious agents such as Hemophilus, Cryptococcus, Streptococcus, Hepatitis B virus, HIV, Lyme disease, and Chlamydia trichomatis. These immunoassay tests are commonly used to identify patients with these and other diseases. Accordingly, compositions and methods for improving the sensitivity, specificity, and detection limits in immunoassays are of great importance in the field of diagnostic medicine.

The term “analyte” refers to a substance or chemical constituent whose presence or absence in, for example, a biological sample is being determined via an immunoassay or other diagnostic test.

“Biomolecule” as used herein refers without limitation to a nucleic acid (e.g., DNA or RNA) or a protein (e.g., an antibody) but further includes any organic molecule removed from an organism (e.g., a human patient).

The following examples are offered by way of illustration and not by way of limitation:

### EXAMPLES

### Example 1

### Grafting of GMA to a Nitrocellulose Membrane

GMA was grafted to a nitrocellulose membrane (e.g., an un-backed nitrocellulose membrane (“NC”) or a polyester (PE)-backed nitrocellulose membrane (“PE-backed NC”) at either the three or six carbon position on the nitrocellulose backbone using an aqueous solution comprising an 8% GMA (v/v) solution in Tween-20™ (polyoxyethylene (20) sorbitan monolaurate). The membranes were slowly dipped into the GMA solution to saturate the membrane, and the excess solution was removed. The nitrocellulose membranes were then exposed to e-beam radiation using an EBLAB-150 (Advance Electron Beams, Wilmington, Mass.) at a dosage of 10 kGy or 50 kGy at 125 kV with the nitrocellulose membrane passing under the e-beam at 50 feet per minute. The membrane was washed three times in deionized water and then agitated in several changes of deionized water for 1-2 hours. The nitrocellulose membrane was dried overnight at 50° C. and 25 mm Hg, and the weight gain after GMA grafting was determined. In certain experiments, the nitrocellulose membranes were first exposed to e-beam radiation and then dipped in the aqueous GMA solution, but the remainder of the experiments were performed using this grafting process was carried out as set forth above. Those experiments in which the nitrocellulose membrane was first irradiated are indicated in the tables provided herein below.

The percentage weight gain (e.g., relative to that of an unmodified membrane) of the un-backed (“NC”) and PE-backed (“PE-backed NC”) nitrocellulose membranes following GMA grafting at various e-beam radiation doses is provided in Table 1. The weight gain is expressed as the percentage weight gain relative to that of unmodified un-backed or PE-backed nitrocellulose membrane, as appropriate.

TABLE 1
Weight Gain of Nitrocellulose Membranes
Following Grafting with GMA

| | | | Weight gain | |
|---|---|---|---|---|
| | | | after wash/dry | |
| # | GMA | E-beam | PE-backed NC | NC |
| 1 | 8% GMA | E-beam (50 kGy) → Dipping | 5.3% | 26.5% |
| 2 | Tween ™ | Dipping → E-beam (50 kGy) | 6.8% | 34.1% |
| 3 | 20 Water | Dipping → E-beam (20 kGy) | 6.3% | 31.6% |
| 8 | | Dipping → E-beam (15 kGy) | 6.0% | 30% |
| 9 | | Dipping → E-beam (10 kGy) | 6.6% | 32.8% |

The increase in weight following the GMA grafting process set forth above supports the successful introduction of GMA on the nitrocellulose membranes. The weight gain following grafting of GMA of both the un-backed and PE-backed nitrocellulose membranes was greater when the membranes were first saturated with the aqueous GMA solution prior to e-beam irradiation, and, therefore, this ordering of the steps for grafting GMA onto a nitrocellulose membrane was used in all later experiments. A slight increase in weight gain was observed when the nitrocellulose membranes were grafted with GMA at an e-beam dosage of 50 kGy versus 10 kGy, as shown in Table 1.

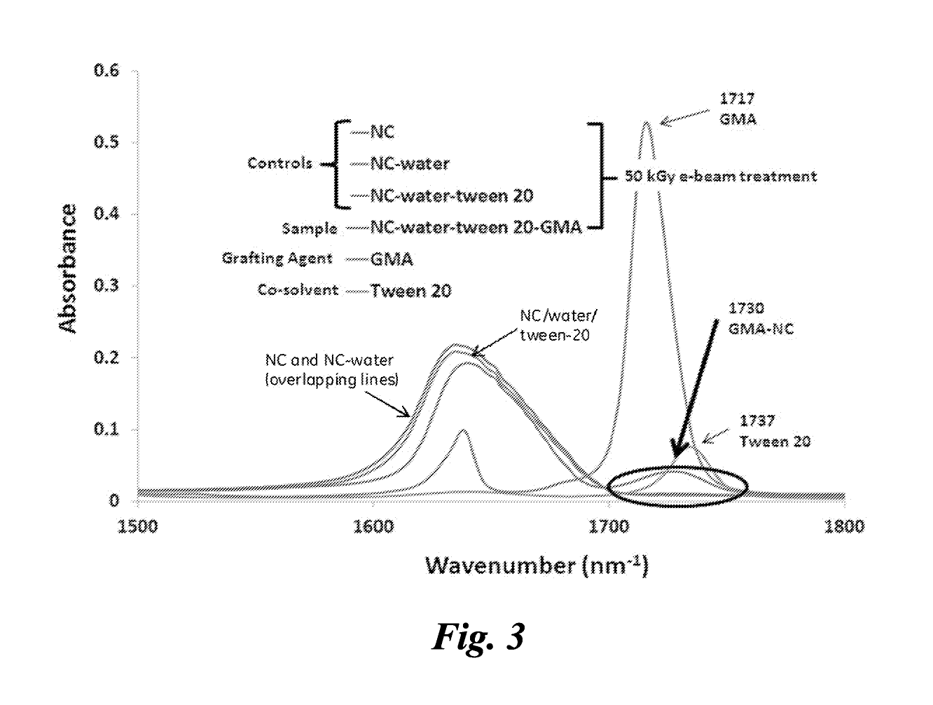

To further confirm the successful grafting of GMA on the porous membranes, nitrocellulose membranes were analyzed by ATR FT-IR using a PerkinElmer Spectrum 100 FTIR spectrophotometer (PerkinElmer Life and Analytical Sciences, Sheraton, Conn.). In ¹H-NMR, the carboxylic peak in GMA and in Tween-20™ (polyoxyethylene (20) sorbitan monolaurate) as individual components appears at 1717 and 1737 nm⁻¹, respectively. The generation of a carboxylic group as a result of GMA introduction yields a peak around 1730 cm⁻¹ on the spectrum in the nitrocellulose-GMA samples. No Tween-20™ (polyoxyethylene (20) sorbitan monolaurate) was detected in the nitrocellulose-GMA samples by ATR FT-IR. See FIG. 3.

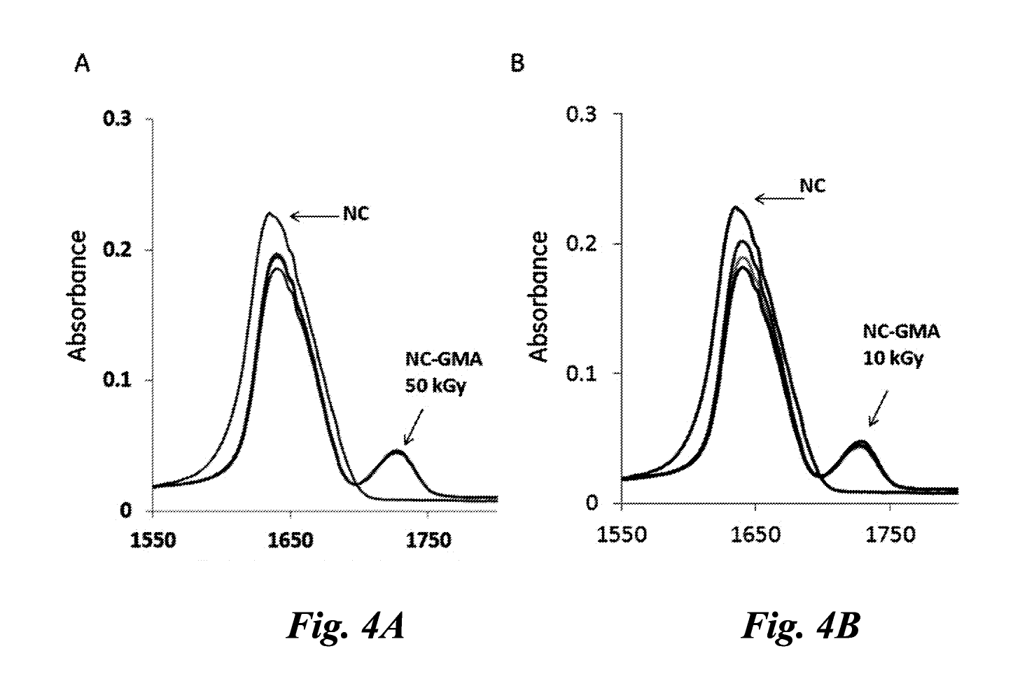

The reproducibility of the GMA grafting process set forth above was verified by measuring the percent weight gain of multiple un-backed and PE-backed nitrocellulose membrane samples following grafting with GMA at e-beam dosages of 10 kGy or 50 kGy. The results are presented in Table 2 and demonstrate that the amount of GMA grafted to either an un-backed or PE-backed nitrocellulose membrane is consistent and reproducible at both 10 kGy and 50 kGy of e-beam radiation. The consistent results observed with GMA grafting of nitrocellulose membranes were further verified by ATR-FTIR as described above. The carboxylic peak heights of different samples grafted with GMA under the same experimental conditions are identical. See FIG. 4.

TABLE 2
Reproducible Weight Gain of Nitrocellulose
Samples After GMA Grafting

| | |
|---|---|
| E-beam | Weight gain (%) |

| dosage | Sample | PE-backed NC | NC |
|---|---|---|---|
| 50 kGy | 1 | 7.1 | 35.5 |
| | 2 | 6.6 | 33.2 |
| | 3 | 7.1 | 35.3 |
| | 4 | 6.5 | 32.3 |
| 10 kGy | 1 | 7.1 | 35.3 |
| | 2 | 6.3 | 31.7 |
| | 3 | 6.3 | 31.6 |

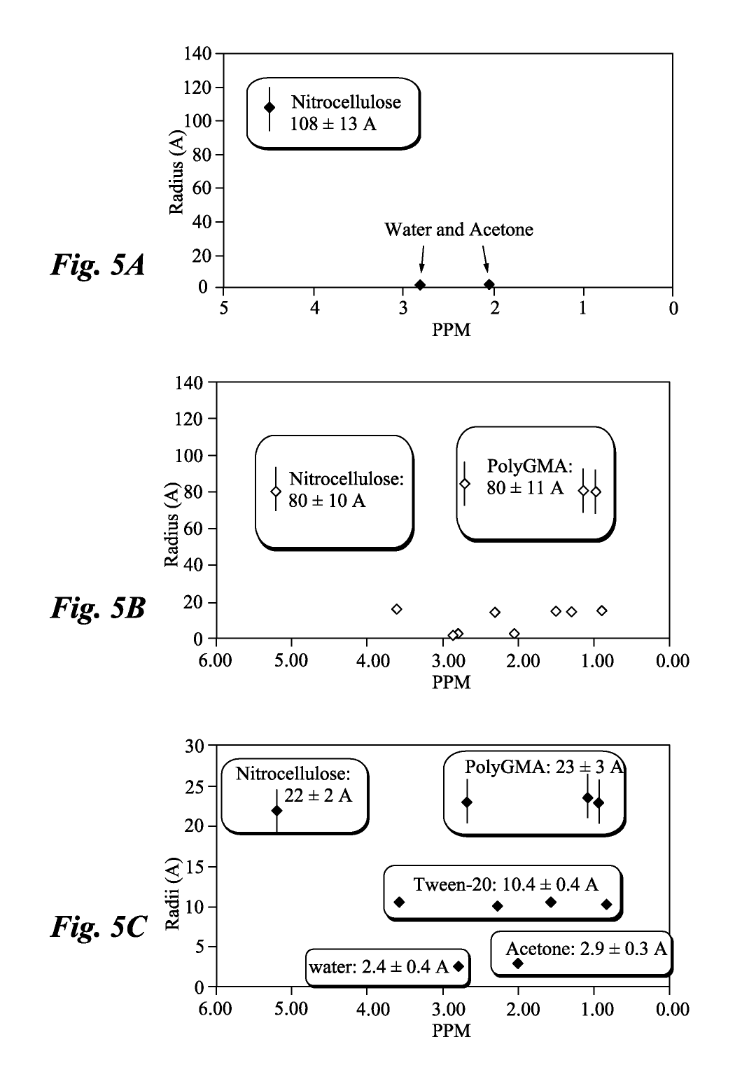

Initial NMR analysis indicated the self-polymerization of GMA on the nitrocellulose membranes. The covalent linkage between GMA and the nitrocellulose backbone was further verified by DOSY NMR. ¹H DOSY NMR analysis of acetone dissolved nitrocellulose samples, with or without GMA grafting at either 10 kGy or 50 kGy of e-beam radiation, were run on a Varian NMRS 600 spectrometer using a 5 mm indirect detect triple-axis gradient probe. As shown in FIG. 5A, untreated nitrocellulose has a hydrodynamic radius of 108±13 Å. For the nitrocellulose sample grafted with GMA at an e-beam dosage of 50 kGy e-beam radiation, the hydrodynamic radius of the nitrocellulose moiety is around 22±2 Å, which is identical to that of the GMA moiety (23±3 Å). See FIG. 5B. The hydrodynamic radius of the nitrocellulose moiety for the nitrocellulose sample grafted with GMA at an e-beam dosage of kGy is 80±10 Å, which is identical to that of the GMA moiety (80±11 Å). See FIG. 5C. Trace amounts of Tween-20™ (polyoxyethylene (20) sorbitan monolaurate) were detected by DOSY NMR in nitrocellulose samples grafted with GMA at e-beam dosages of both 10 kGy and 50 KGy.

The observation that the nitrocellulose and the GMA polymer moieties have the same hydrodynamic radius independent of the e-beam dosage used in the process of grafting GMA onto nitrocellulose indicates that the GMA is covalently linked to the nitrocellulose.

### Example 2

### Properties of Modified Nitrocellulose Membranes Grafted with GMA

The modified nitrocellulose membranes grafted with GMA as described above were further characterized to assess membrane thickness, capillary rise, and mechanical strength (e.g., stress and strain). The modified nitrocellulose membranes were grafted with GMA as described in Example 1 using a 10 kGy or 50 kGy e-beam dosage.

Capillary rise was tested using an Ontario Die 10 mm by 50 mm notched punch to cut test strips from unmodified and modified, GMA-grafted nitrocellulose membranes. The test strips were placed in a device to keep the test membrane strips vertical. The device also has a shallow groove to hold the test fluid (e.g., distilled water). The rise time of 40 μL of distilled water to the notch at a height of 40 mm was recorded. The capillary rise of replicate samples of unmodified and modified nitrocellulose membranes was measured. The results are summarized below in Table 3.

As set forth in Table 3, the modified nitrocellulose membranes grafted with GMA exhibited a 15-20% increase in membrane thickness and an approximately 10% slower capillary flow rate relative to the unmodified nitrocellulose membranes. Moreover, although e-beam treatment resulted in membrane degradation of the modified nitrocellulose membranes, the polymers of GMA grafted to these nitrocellulose membranes improved their mechanical strength (e.g., stress and strain).

TABLE 3
Characterization of Modified Nitrocellulose Membranes Grafted with GMA

| | | Capillary Rise | | Mechanical strength (AE98 fast) | |
|---|---|---|---|---|---|
| | | (seconds to rise 4 cm) | Membrane | Stress at Max | Strain at Max |
| Characterization | | (PE-backed NC) | Thickness (μm) | load (kgf/cm²) | load (%) |
| NC, unmodified | | 97.1 ± 1.7 | 102.9 ± 0.6 | 62.5 ± 4.8 | 4.8 ± 1.1 |
| NC- | 10 kGy | 128.1 ± 20.8 | 113.5 ± 3.5 | 87.0 ± 12.1 | 12.1 ± 0.2 |
| GMA | 50 kGy | 112.3 ± 4.0 | 119.5 ± 1.9 | 85.4 ± 3.1 | 12.9 ± 3.2 |

Mechanical strength testing was performed using an Instron Universal Testing Instrument Model 4202 (Norwood, Mass.). Specifically, stress and strain were measured on unmodified and modified nitrocellulose membrane samples with a 6.5 mm width and a gauge length of 51 mm. The test rate was 25 mm per minute. The mechanical strength of replicate samples of unmodified and modified nitrocellulose membranes was measured. The results are summarized below in Table 3.

### Example 3

### Protein Binding to Nitrocellulose Membranes Grafted with GMA

The ability of both unmodified and modified nitrocellulose membranes grafted with GMA to bind protein under different reaction conditions was determined. In particular, three solutions of BSA were prepared: 1) 5 mg/ml BSA (unlabeled) and 1 mg/ml BSA-FITC in PBS at pH 7.4; 2) 5 mg/ml BSA (unlabeled) and 1 mg/ml BSA-FITC in sodium phosphate at pH 8.0; and 3) 1 mg/ml BSA-FITC in sodium phosphate at pH 8.0. Unmodified or modified GMA-grafted nitrocellulose membranes were incubated in the above BSA solutions for 2 hours or 15 hours at 30° C. The membranes were then washed with a 0.5% Tween-20™ (e.g., polyoxyethylene (20) sorbitan monolaurate) solution to remove non-specific binding of the BSA to the nitrocellulose membrane and then briefly rinsed with deionized water.

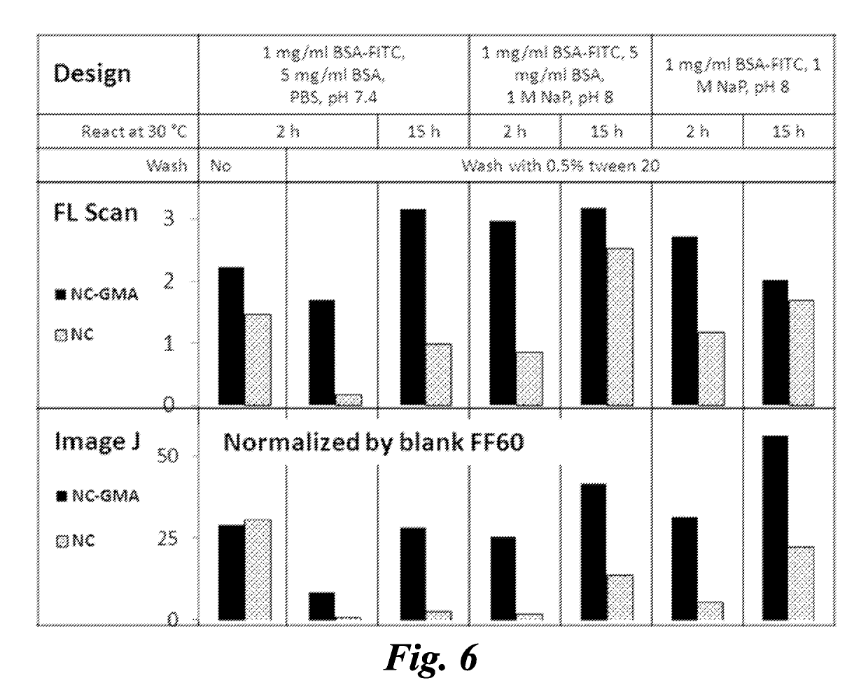

The nitrocellulose membranes were analyzed by both fluorescence scanning and colorimetric analysis to assess protein binding. The fluorescence scanning analysis was performed using a GE Typhoon 9400 Fluorescence Scanner with excitation/emission wavelengths of 485 nm and 520 nm, respectively. Colorimetric analysis of the nitrocellulose membranes was performed using the publically available ImageJ processing program, using an unmodified nitrocellulose membrane to serve as the base-line. The results of these analyses are shown in FIG. 6.

To summarize, the modified nitrocellulose membranes grafted with GMA retained 2-3 fold more protein relative to the amount of protein able to bind to the unmodified nitrocellulose membranes. This result was observed with all of the different reaction conditions tested. Furthermore, increasing the ionic strength and pH of the reaction buffer improved the protein binding efficiency such that detection times of the bound protein were significantly reduced (e.g., 15 hours to 2 hours).

### Example 4

### Use of Modified Nitrocellulose Membranes in Lateral Flow Assays

Lateral flow assays that require the immobilization of a protein, more particularly an antibody, on a solid phase material form the basis of a number of in vitro diagnostic tests. One common example of this technology is commercially available home pregnancy tests which rely on the immobilization of an antibody that recognizes human chorionic gonadotropin (hCG), a hormone that is produced in high levels during pregnancy. Techniques to assess the utility of the modified nitrocellulose membranes grafted with GMA in lateral flow assays were designed based on this pregnancy test model.

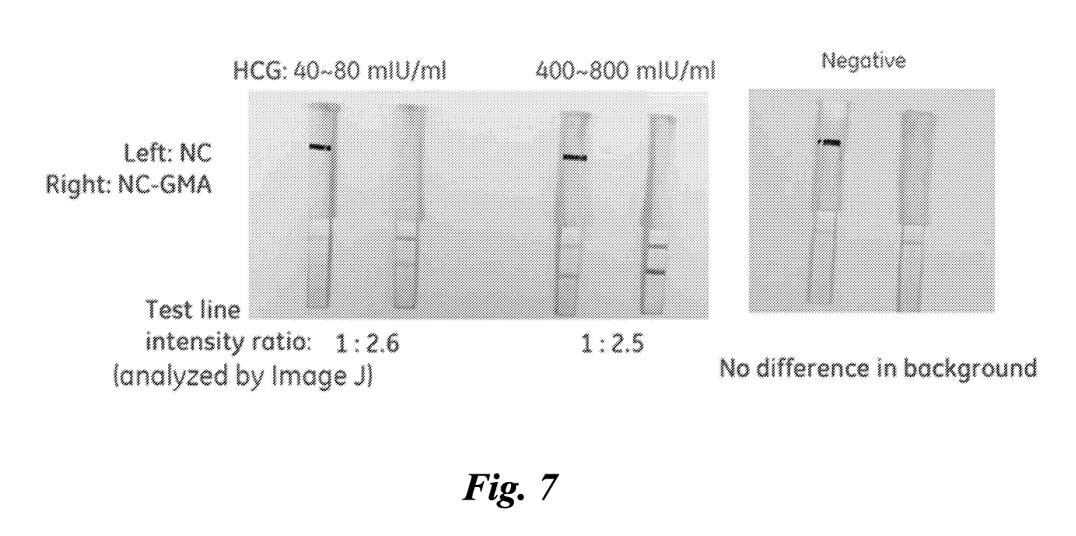

A control and a test line were deposited on unmodified or modified nitrocellulose membranes grafted with GMA using inkjet printing in accordance with standard techniques in the art. A basic inkjet formulation containing glycerol, Triton X-100, and CMC was used to prepare the control line further contained 0.5 mg/ml goat anti-mouse IgG. The ink for the test line additionally contained 1 mg/ml of an anti-HCG-α antibody. 24 hours after the control and test lines were printed onto the modified nitrocellulose membranes the membranes were laminated onto a G&L polyester backing pre-treated with GL 187 glue. A 27 mm Whatman CF₇ absorbent pad was then laminated on top of the nitrocellulose membranes. The membranes were cut using a 5 mm×10 mm punch into strips 5 mm in length. The strips were then dipped into 100 μl of running buffer containing various Concentrations of hCG (0, 40-80, or 400-800 mIU/ml), 0.5% Tween-20™ (polyoxyethylene (20) sorbitan monolaurate) as a blocking agent, and a gold nanoparticle (AuNP)-anti-HCG-β antibody conjugate as the reporting agent. After 20-30 minutes, the assay was completed. The colorimetric reporting signal intensity was assessed both by visual inspection and by ImageJ analysis to obtain a quantitative comparison. The results obtained with the lateral flow assays using unmodified (NC) or modified nitrocellulose membranes grafted with GMA (NC-GMA) are presented in FIG. 7.

The colorimetric reporting signal was visible within approximately 3-5 minutes in the assays performed with the unmodified nitrocellulose membranes and within about only 1 minute in those assays that used the modified GMA-grafted nitrocellulose membranes. A 250% increase in signal intensity of the test line was observed on the modified nitrocellulose membranes grafted with GMA relative to that on the unmodified nitrocellulose membranes. No difference in the background signal was observed between the unmodified and modified nitrocellulose membranes during the time frame of the assay.

### Example 5

### Decreased Detection Limit Requirements of hCG Using Modified Nitrocellulose Membranes in Lateral Flow Assays

Unmodified or modified nitrocellulose membrane strips were printed with an inkjet printer with a first antibody that specifically binds to hCG-α. The unmodified or modified nitrocellulose membrane strips were assembled into 5 mm half-stick lateral flow devices. Running buffer was prepared by mixing different concentrations of hCG samples with a second antibody that specifically binds to anti-hCG-β. The anti-hCG-β antibodies were conjugated to the detectable substance gold nanoparticles. The running buffer contained a range of concentrations of hCG of approximately 0.1 to 500 mIU/ml. The running buffer further comprised 0.5% Tween-20™ (e.g., polyoxyethylene (20) sorbitan monolaurate).

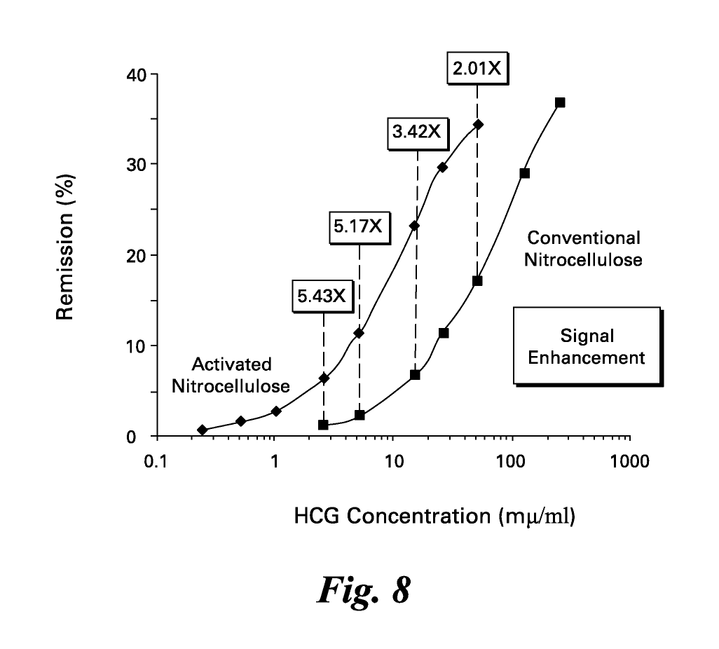

The 5 mm half-stick lateral flow devices were dipped in 100 μl running buffer, and the assay was completed within thirty (30) minutes. The results obtained with the unmodified and the modified nitrocellulose membranes grafted with polymers of GMA were quantified using an LRE colorimetric reflectance reader to assess antigen-antibody binding. The results demonstrate improved signal intensity across the entire hCG concentration range. Moreover, the increased signal intensity was detected at hCG levels significantly lower than those obtained in corresponding examples using unmodified nitrocellulose membrane strips. Specifically, the detection limit is 0.5 mIU/ml for the half-stick made from the modified nitrocellulose grafted with GMA, which is more than 5-fold lower than that observed in the same methods instead using an unmodified nitrocellulose membrane strip. These results provide strong evidence that immunoassays performed with a modified nitrocellulose membrane comprising a polymer coating of, for example, GMA, notably lowered the amount of analyte or biomolecule necessary in the sample for detection of an antigen of interest. See FIG. 8.

### Example 6

### Decreased Detection Time of hCG Using Modified Nitrocellulose Membranes in Lateral Flow Assays

The kinetics of signal development in the pregnancy lateral flow assay, essentially as described above, were performed using testing devices comprising unmodified nitrocellulose membranes and those with modified grafted with a GMA polymer coating. These analyses were performed by dipping the lateral flow device into a running buffer containing 150 mIU/ml hCG, gold nanoparticles labeled anti-hCG-beta IgG, and 0.5% tween 20. Images were taken at different time points for 30 minutes, the signal line intensities on the lateral flow devices were measured by Image J software.

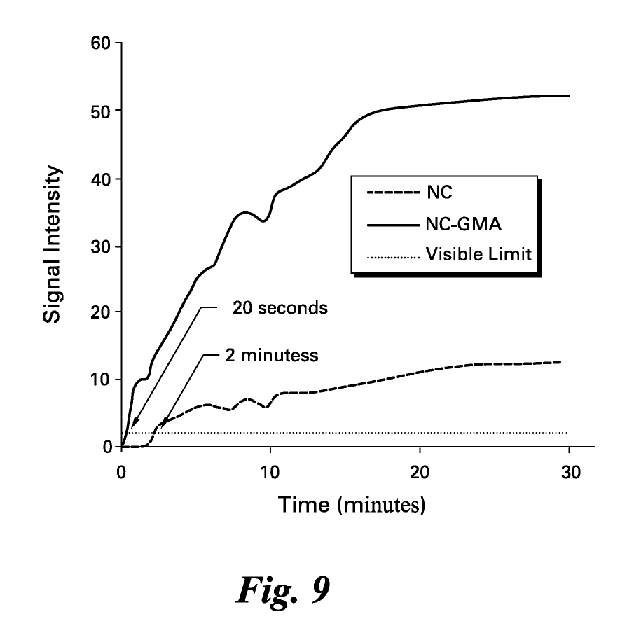

The results are presented in FIG. 9 demonstrate that it took 20 seconds to visualize a positive signal (e.g., for the presence of hCG) when the assays were performed using modified nitrocellulose membranes grafted with a GMA polymer coating. In contrast, a detectable result was obtained at two minutes when the lateral flow assays were performed with unmodified nitrocellulose membranes, six-times longer than was required to visualize a the test result when the lateral flow device comprises a nitrocellulose membrane with a polymer coating of GMA. Lowered detection times present an obvious advantage in in vitro diagnostics in which timing of diagnosis may be critical to patient outcome and also in those situations (e.g., home pregnancy tests) in which significantly faster detection time is also desirable.

While only certain features of the invention have been illustrated and described herein, many modifications and changes will occur to those skilled in the art. It is, therefore, to be understood that the appended claims are intended to cover all such modifications and changes as fall within the true spirit of the invention.

All publications, patent publications, and patents are herein incorporated by reference to the same extent as if each individual publication or patent was specifically and individually indicated to be incorporated by reference.

1. A method for improving detection of a biomolecule in a biological sample using an immunoassay comprising:
a) providing a modified porous membrane having the structure of Formula (I), wherein Formula (I) is: 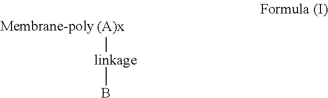
wherein A is an electron beam (e-beam) reactive moiety, wherein poly (A)ₓ is a polymer of the e-beam reactive moiety and x is a number of A monomers present in the poly (A)ₓ polymer; wherein the linkage forms a bond between the poly (A)ₓ polymer and a B group, and wherein the poly(A)ₓ-linkage-B is a polymer coating covalently grafted to the porous membrane;
b) incubating the modified porous membrane in the biological sample that may contain the biomolecule;
c) washing the modified porous membrane to remove unbound material;
d) measuring biomolecule binding to the modified porous membrane and biomolecule binding to the unmodified porous membrane; and
e) comparing biomolecule binding to the modified porous membrane with biomolecule binding to the unmodified porous membrane, thereby improving detection of the biomolecule in the biological sample.

2. The method of claim 1, wherein the biomolecule exhibits improved immobilization on the modified porous membrane relative to immobilization of the biomolecule on the unmodified porous membrane.

3. The method of claim 1, wherein the membrane is selected from the group consisting of a nitrocellulose membrane, a cellulose membrane, a cellulose acetate membrane, a regenerated cellulose membrane, a nitrocellulose mixed ester membranes, a polyethersulfone membrane, a nylon membrane, a polyolefin membrane, a polyester membrane, a polycarbonate membrane, a polypropylene membrane, a polyvinylidene difluoride membrane, a polyethylene membrane, a polystyrene membrane, a polyurethane membrane, a polyphenylene oxide membrane, a poly(tetrafluoroethylene-co-hexafluoropropylene membrane, and any combination of two or more of the above membranes.

4. The method of claim 3, wherein the porous membrane is a nitrocellulose membrane.

5. The method of claim 1, wherein the e-beam reactive moiety of A is selected from the group consisting of a methacrylate, an acrylate, an acrylamide, a vinyl ketone, a styrenic, a vinyl ether, a vinyl-containing moiety, an allyl-containing moiety, a benzyl-based compound, a tertiary-carbon (CHR₃)-based compound, and any combination of two or more of the above functional moieties.

6. The method of claim 1, wherein the linkage is an ester, an aliphatic, an aromatic, a hydrophilic compound, a hetero-aromatic compound, or any combination of two or more of the above linkages.

7. The method of claim 1, wherein the B group is selected from the group consisting of an epoxy group-containing compound, a polyethylene glycol (PEG), an alkyne group, a hydroxyl group, an amine group, a halogen group, a tosyl group, a mesyl group, an azido group, an isocyanate group, an silane group, disilazanes, sulfhydryls, carboxylates, isonitriles, phosphoramidites, nitrenes, hydrosilyl, nitrile, alkylphosphonates, and any combination of two or more of the above functional moieties.

8. The method of claim 7, wherein the B group is an epoxy group-containing compound.

9. The method of claim 8, wherein the epoxy group-containing compound is glycidal methylacrylate (GMA), glycidal acrylate, vinyl glycidyl ether, allyl glycidyl ether, methallyl glycidyl ether, or any combination thereof.

10. The method of claim 9, wherein the epoxy group-containing compound is GMA.

11. The method of claim 1, wherein the biomolecule is DNA, RNA, or a protein.

12. The method of claim 11, wherein the biomolecule is a protein.

13. The method of claim 12, wherein the protein is an antibody.

14. The method of claim 1, wherein measuring biomolecule binding comprises labeling with a detectable substance the biomolecule or a molecule that specifically binds to the biomolecule.

15. The method of claim 14, wherein the detectable substance is an enzyme, a prosthetic group, a fluorescent dye, a luminescent material, a bioluminescent material, a radioactive material, or a gold particle.

16. The method of claim 15, wherein the detectable substance provides quantitative results as to biomolecule level.

17. The method of claim 15, wherein the detectable substance provides qualitative results as to biomolecule level.

18. The method of claim 1, wherein the washing of the modified porous membrane is performed in a solution further comprising a non-ionic surfactant, and wherein the non-ionic surfactant is polyoxyethylene (20) sorbitan monolaurate.

19. The method of claim 1, wherein the immunoassay is selected from the group consisting of a lateral flow immunoassay, a radioimmunoassay, an enzyme immunoassay (EIA), an enzyme-linked immunosorbent assay (ELISA), a fluorescent immunoassay, and a chemiluminescent immunoassay.

20. The method of claim 1, wherein the biological sample is blood, serum, lymph, urine, saliva, mucus, bodily secretions, cells, or tissue.

21. The method of claim 1, wherein the method is performed with the porous membrane on a solid support, and wherein the solid support is a microtiter plate or a glass slide.

22. The method of claim 1, wherein the method permits the detection of more than one biomolecules in the biological sample.

23. The method of claim 1, wherein the biomolecule is an antigen associated with a disease.

24. The method of claim 23, wherein the disease is a bacterial disease, a viral disease, or a fungal disease.

25. A method for decreasing biomolecule level needed to detect presence of the biomolecule in a biological sample comprising:
a) providing a modified porous membrane having the structure of Formula (I), wherein Formula (I) is: 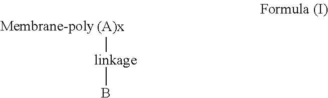
wherein A is an e-beam reactive moiety, wherein poly (A)ₓ is a polymer of the e-beam reactive moiety and x is a number of A monomers present in the poly (A)ₓ polymer; wherein the linkage forms a bond between the poly (A)ₓ polymer and a B group, and wherein the poly(A)ₓ-linkage-B is a polymer coating covalently grafted to the porous membrane;
b) incubating the modified porous membrane in the biological sample to determine if the biological sample comprises the biomolecule;
c) washing the modified porous membrane to remove unbound material; and
d) measuring biomolecule binding to the modified porous membrane and biomolecule binding to the unmodified porous membrane, wherein the biomolecule exhibits improved immobilization to the modified porous membrane, and thereby lowers the amount of biomolecule needed to detect the biomolecule in a biological sample.

26. The method of claim 25, wherein the biomolecule is DNA, RNA, or a protein.

27. The method of claim 26, wherein the biomolecule is a protein.

28. The method of claim 27, wherein the protein is an antibody.

29. A method of improving immobilization of a biomolecule on a porous membrane comprising:
a) providing a modified porous membrane having the structure of Formula (I), wherein Formula (I) is: 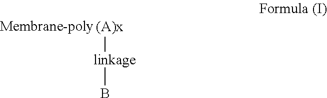
wherein A is an e-beam reactive moiety, wherein poly (A)ₓ is a polymer of the e-beam reactive moiety and x is a number of A monomers present in the poly (A)ₓ polymer; wherein the linkage forms a bond between the poly (A)ₓ polymer and a B group, and wherein the poly(A)ₓ-linkage-B is a polymer coating covalently grafted to the porous membrane;
b) incubating the modified porous membrane in a sample comprising the biomolecule;
c) washing the modified porous membrane to remove unbound material;
d) measuring biomolecule binding to the modified porous membrane and biomolecule binding to the unmodified porous membrane; and
e) comparing biomolecule binding to the modified porous membrane with biomolecule binding to the unmodified porous membrane, wherein immobilization of the biomolecule on the modified porous membrane is improved relative to immobilization of the biomolecule on the unmodified porous membrane.

30. The method of claim 29, wherein the biomolecule is a protein.

31. A method for reducing detection time to determine presence of a biomolecule in a biological sample using an immunoassay comprising:
a) providing a modified porous membrane having the structure of Formula (I), wherein Formula (I) is: 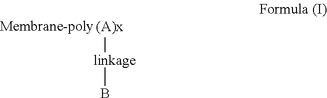
wherein A is an e-beam reactive moiety, wherein poly (A)ₓ is a polymer of the e-beam reactive moiety and x is a number of A monomers present in the poly (A)ₓ polymer; wherein the linkage forms a bond between the poly (A)ₓ polymer and a B group, and wherein the poly(A)ₓ-linkage-B is a polymer coating covalently grafted to the porous membrane;
b) incubating the modified porous membrane in the biological sample that may contain the biomolecule;
c) washing the modified porous membrane to remove unbound material;
d) measuring biomolecule binding to the modified porous membrane and biomolecule binding to the unmodified porous membrane; and
e) comparing biomolecule binding to the modified porous membrane with biomolecule binding to the unmodified porous membrane at various time points, wherein the biomolecule binds more quickly to the modified porous membrane, thereby reducing the time needed to detect the presence of the biomolecule in the biological sample.

32. A method for reducing antibody amount needed to perform an immunoassay that uses a porous membrane for immobilization of the antibody, wherein the porous membrane used in the immunoassay has the structure of Formula (I), wherein Formula (I) is: 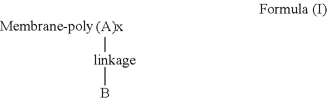
wherein the antibody exhibits improved immobilization to the porous membrane of Formula (I).

33. The method of claim 32, wherein reducing the amount of antibody needed to perform the immunoassay reduces cost of performing the immunoassay.

34. A method for improving the sensitivity of an immunoassay that uses a porous membrane for immobilization of a biomolecule, wherein the porous membrane used in the immunoassay has the structure of Formula (I), wherein Formula (I) is:

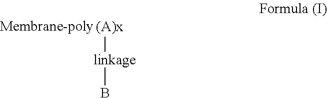

35. The method of claim 34, wherein the biomolecule is a protein.

36. The method of claim 35, wherein the protein is an antibody.
